(19) United States
(12) Patent Application Publication
(10) Pub. No.: US 2016/0000722 A1
(43) Pub. Date: Jan. 7, 2016

(54) DOSAGE FORM FOR CINEOLE

(71) Applicants: Harald Greve, Dusseldorf (DE); Walter Duchatsch, Siegburg (DE)

(72) Inventors: Harald Greve, Dusseldorf (DE); Walter Duchatsch, Siegburg (DE)

(21) Appl. No.: 13/697,463

(22) PCT Filed: Apr. 13, 2011
(86) PCT No.: PCT/EP2011/001862

§ 371 Date: Aug. 10, 2015

(30) Foreign Application Priority Data
May 12, 2010 (DE) 10 2010 020 425.0
May 21, 2010 (DE) 10 2010 022 174.0

(51) Int. Cl. A61K 9/48; A61K 9/50; A61K 45/06; A61K 31/352; A61K 9/00

(52) U.S. Cl. CPC A61K 9/4808; A61K 31/352; A61K 9/0053; A61K 45/06; A61K 9/4833; A61K 9/4858; A61K 9/4866; A61K 9/4891; A61K 9/5089

(57) ABSTRACT

The invention relates to a dosage form containing cineole for peroral application in capsule form, wherein the dosage form containing cineole is designed as a capsule-in-capsule system, wherein the capsule-in-capsule system has an outer capsule (outside capsule) having an outer capsule shell and a plurality of inner capsules (inside capsules) located in the outer capsule, wherein the inner capsules are completely enclosed by the capsule shell of the outer capsule and the inner capsules are designed as microcapsules, which each contain the active ingredient 1,8-cineole. The invention further relates to the production and use of said dosage form containing cineole.

### CROSS-REFERENCES TO RELATED APPLICATIONS

This application is a National Stage filing of International Application PCT/EP 2011/001862, filed Apr. 13, 2011, claiming priority to German Applications No. DE 10 2010 020 425.0 filed May 12, 2010, and DE 10 2010 022 174.0 filed May 21, 2010, entitled “NEW DOSAGE FORM FOR CINEOLE.” The subject application claims priority to PCT/EP 2011/001862, and to German Applications No. DE 10 2010 020 425.0, and DE 10 2010 022 174.0, and incorporates all by reference herein, in their entirety.

### BACKGROUND OF THE INVENTION

The present invention relates to a cineole-containing dosage form, designed in the form of a capsule, for peroral application and to a process for its production and its use, particularly in the field of human and veterinary medicine. Furthermore, the present invention relates to a drug or medicinal product comprising the cineole-containing dosage form according to the invention.

1,8-Cineole belongs to the bicyclic epoxy-monoterpenes, more precisely expressed the limonene oxides. Synonymous names for 1,8-cineole with the chemical empirical formula C₁₀H₁₈O are eucalyptol, limonene 1,8-oxide, 1,8-epoxy-p-menthane or 1,3,3-trimethyl-2-oxobicyclo[2.2.2]octane. It is a colorless liquid with a spicy, camphor-like odor with a melting point of +1.5° C. and a boiling point of 176 to 177° C., which is insoluble in water but miscible with most organic solvents.

Naturally, 1,8-cineole occurs as the main constituent of eucalyptus oil (eucalyptus oil contains up to 85% by weight of 1,8-cineole), but also in other plants, thus e.g. in mint, medicinal sage, thyme, basil and in tea tree. Moreover, 1,8-cineole is present for example in niaouli oil, juniper oil, piper oil, cannabis oil, cajuput oil, sage oil, myrtle oil and other essential oils.

Industrial 1,8-cineole, which is generally 99.6 to 99.8% pure, is generally obtained by fractional distillation of eucalyptus oil.

1,8-Cineole is used in particular as expectorant in bronchial catarrhs and other respiratory tract diseases primarily in veterinary medicine, but moreover also as aroma substance in the perfume industry. Moreover, 1,8-cineole is used in dental medicine in the revision of root fillings.

From a physiological point of view, 1,8-cineole has a mucolytic and bactericidal effect in the upper and lower respiratory tracts, especially in the lungs and the sinuses. Furthermore, it inhibits certain neurotransmittens which are responsible for the narrowing of bronchi. In chronic-obstructive lung diseases and bronchial asthma, the lung function can be improved by administering pure 1,8-cineole. On account of its corticosteroid-like effect, in the case of serious respiratory tract diseases, it is used as a substitute to or in comedication with corticosteroids with systemic application. 1,8-Cineole can in principle be used both topically, e.g. inhalatively, or else systemically, especially in the form of capsules.

As active ingredient, 1,8-cineole thus has mucolytic and anti-inflammatory effects. When used systemically, 1,8-cineole is easily absorbed and passes via the bloodstream to take effect in the respiratory organs. In this way, 1,8-cineole can for example liquefy inflammatory secretions and viscous mucous in the airways and counteract inflammatory processes in the respiratory tracts. A buildup of secretion is thus prevented which facilitates expectoration, supports the function of the cilia responsible for cleaning in the bronchi and the nose and thus improves the aeration of the respiratory tracts. In the region of the upper airways, the hindering of nasal breathing during colds and the heaviness of the head disappear.

For further details relating to the active ingredient 1,8-cineole, reference may be made for example to Römpp Chemistry Lexicon, Georg Thieme Verlag, Stuttgart/New York, 10^{th} edition, volume 1, 1996, page 752, keyword: “Cineole”, and the literature referred to therein.

In the documents DE 43 19 554 C2, DE 43 19 556 C2 and WO 94/28895 A2, which belong to the same patent family, a combination therapy with perorally administered terpene compounds, in particular 1,8-cineole or menthol, on the one hand and likewise systemically, in particular perorally administered corticosteroids on the other hand is described for the anti-inflammatory treatment of systemically steroid-dependent chronic bronchial asthma, where the use of the perorally administered terpene compounds in the course of a long-term treatment is said to reduce the need for systemic corticosteroids. Preference is given to using gastric juice-resistant, but small intestine-soluble capsules with the terpene-based active ingredient.

For the systemic application of 1,8-cineole, various preparations, in particular based on generally gastric juice-resistant, but small intestine-soluble capsules, are commercially available, with both monopreparations, which comprise 1,8-cineole as the sole active ingredient and/or in pure form, and also so-called combination preparations, which comprise 1,8-cineole in a complex mixture with a multitude of further terpenes, being available and/or known.

In most commercially available capsules with 1,8-cineole as active ingredient, an optimum long-term stabilization or an optimum protection against the surrounding milieu, in particular against atmospheric oxidation, is generally not always present. Furthermore, with the cineole-containing capsule systems known from the prior art, a controlled or retarded or delayed release is often not optimized and/or not always possible. Similarly, in the case of the cineole-containing active ingredient capsules known from the prior art, an optimal dosage of the active ingredient is often also not always possible. Moreover, with the cineole-containing capsule systems known from the prior art, optimal taste and/or odor masking is often not possible. Also, side effects occasionally arise, especially in the case of relatively high doses, such as gastrointestinal troubles, in particular gastric heaviness, reflux, nausea, diarrhea or the like.

### BRIEF SUMMARY OF THE INVENTION

The object of the present invention is therefore to provide an efficient dosage form, intended for peroral application, for 1,8-cineole as active ingredient and a corresponding production process for this dosage form, by means of which the disadvantages and side effects described previously and occurring in connection with the prior art are to be at least partially prevented or else at least partially diminished.

In particular, the object of the present invention is to provide an efficient dosage form, intended for peroral application, for 1,8-cineole as active ingredient which permits an improved release profile and/or an improved long-term stabilization with regard to the active ingredient. In particular, the aim here is also to improve the protection of the active ingredient against the surrounding milieu, in particular against oxidation with atmospheric oxygen, and moreover to ensure an improved dosage and/or handling of the active ingredient, in particular also in the course of the production process for the dosage form.

To solve the problem described above, the present invention proposes—according to a first invention aspect—a cineole-containing dosage form for peroral application in capsule form according to the disclosure herein; further, especially preferred and/or advantageous embodiments of this invention aspect are similarly disclosed.

The present invention further provides—according to a second invention aspect—a process for producing the cineole-containing dosage form according to the present invention; further, especially preferred and/or advantageous embodiments of this invention aspect are provided.

In turn, the present invention further provides—according to a third invention aspect—the use of the cineole-containing dosage form according to the present invention further, especially preferred and/or advantageous embodiments of this invention aspect are similarly provided.

Finally, the present invention further provides—according to a fourth invention aspect—a drug or medicinal product; further, especially preferred and/or advantageous embodiments of this invention aspect are similarly provided.

It goes without saying that specific embodiments and configurations which are described only in connection with one invention aspect also apply accordingly with regard to the other invention aspects, even without being expressly described.

In all of the relative or percentage, especially weight-based quantitative data specified below, it should be ensured that these data in the course of the composition according to the invention are to be selected and/or combined by the person skilled in the art such that, in total—optionally taking into consideration further components and/or ingredients and/or additives and/or constituents, in particular as defined below—always 100% or 100% by weight result. Though this goes without saying for the person skilled in the art.

Moreover, it is the case that the person skilled in the art can—especially depending on the use or specific to the individual case—deviate from the quantitative data listed below without departing from the scope of the present invention.

The applicant has now surprisingly found that the problem addressed by the present invention and described at the outset can be solved by providing the active ingredient 1,8-cineole in the context of a capsules-in-capsule system, where the active ingredient 1,8-cineole is present in inner microcapsules of this capsules-in-capsule system such that the 1,8-cineole can be released in a controlled and/or retarded manner following systemic application.

### BRIEF DESCRIPTION OF TEE DRAWINGS

FIG. 1 provides a diagrammatic cross section through a cineole-containing dosage form according to the invention as per one embodiment of the present invention.

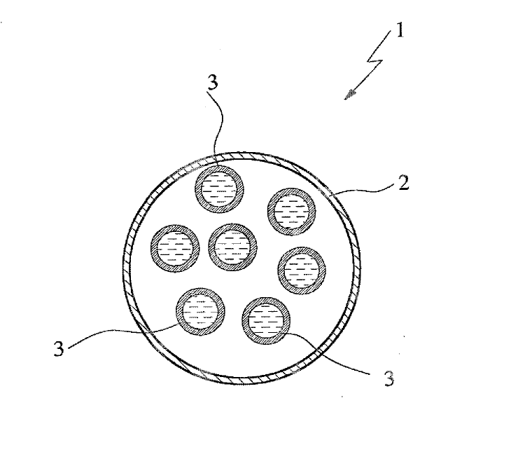

### DETAILED DESCRIPTION OF THE INVENTION

The present invention thus provides—according to a first aspect of the present invention—a cineole-containing dosage form for peroral application in the form of a capsule, where the dosage form is designed as a capsules-in-capsule system, where the capsules-in-capsule system has an outer capsule (outside capsule) having an outer capsule shell and a plurality of inner capsules (inside capsules) located in the outer capsule, where the inner capsules are completely enclosed by the capsule shell of the outer capsule and the inner capsules are designed as microcapsules which each contain the active ingredient 1,8-cineole.

The present invention is associated with a large number of advantages, improvements and special features which distinguish the present invention from the prior art:

Firstly, the dosage form according to the invention based on the above-described capsules-in-capsule system permits a controlled, in particular also retarded or delayed release, such that, for example, there is the possibility of providing the amount of 1,8-cineole to be applied for the daily dose by a single administration, whereas in the case of conventional cineole active ingredient capsules, administration several times spread over the day is required. Secondly, the dosage form according to the invention permits an efficient long-term stabilization of the active ingredient 1,8-cineole, especially also against oxidation for example by atmospheric oxygen. In particular, the active ingredient 1,8-cineole is efficiently protected against the surrounding milieu, such that the storage times are significantly extended compared with conventional cineole capsules. Moreover, the cineole-containing dosage form according to the invention however, also permits an improved taste and/or odor masking of the active ingredient. Moreover, the dosing and mixing with regard to the active ingredient is optimized both during production of the capsules and also upon their use. Furthermore, undesired accompanying phenomena and/or side effects, in particular gastrointestinal troubles, such as, for example, gastric heaviness, overacidification of the stomach, reflux etc., are also significantly reduced and/or avoided entirely.

On the basis of the cineole-containing dosage form according to the invention on the principle of the above-described capsules-in-capsule system, the active ingredient 1,8-cineole is thus effectively stabilized during its storage and protected against ambient influences. On the other hand, an application form is present which permits a controlled release or active ingredient release within the scope of a so-called controlled release effect. In particular, long-term or depot preparations can be provided in this way which release the active ingredient 1,8-cineole over a prolonged period in precisely controlled amounts, in particular release the active ingredient in a targeted manner at the site of action.

In particular, within the context of the present invention, it has for the first time been taken into consideration and for the first time been possible to supply 1,8-cineole in the aforementioned way to a microencapsulation and, on the basis of the microcapsules obtained in this way, to conceive the above-described capsules-in-capsule system which permits an optimum active ingredient dosing and active ingredient use of 1,8-cineole—together with the advantages described above.

By contrast, in the prior art, the provision of the active ingredient 1,8-cineole in microencapsulated form in the context of a capsules-in-capsule system has hitherto neither been contemplated, nor could such a microencapsulation indeed be realized. In particular, in the prior art it has hitherto not been possible to microencapsulate 1,8-cineole in an efficient way, which has only succeeded within the context of the present invention for the first time. This was not to be expected in this way.

As explained above, within the context of the present invention, the active ingredient 1,8-cineole is provided in the context of a capsules-in-capsule system which has an outer capsule (outside capsule) having an outer capsule shell and a plurality of inner capsules (inside capsules) located in the outer capsule, where the inside capsules are completely enclosed by the outside capsule and the inside capsules are designed as microcapsules which each contain the active ingredient 1,8-cineole.

The term microencapsulation as used according to the invention refers in particular to the coating, embedding, etc. of the active ingredient 1,8-cineole with the help of a suitable capsule material, shell material or matrix material, as defined in more detail below.

During the microencapsulation, microcapsules with a defined size and size distribution are produced, as explained below. In principle, a distinction is made here, as is also explained below, between core/shell encapsulation on the one hand and matrix encapsulation on the other hand.

Accordingly, within the context of the present invention, the inside capsules can be present either in the form of matrix capsules or else in the form of core/shell capsules. According to one embodiment preferred according to the invention, the inside capsules are in the form of matrix capsules. Equally, within the context of the present invention, there is the option to combine matrix capsules on the one hand and core/shell capsules on the other hand within a capsules-in-capsule system.

Matrix encapsulation is the terminology used particularly when the active ingredient is present in distributed form over the entire microcapsule or microbead, preferably in homogeneous and/or uniform distribution, whereas in the case of a core/shell encapsulation, the active ingredient is located in the interior or in the cavity of a capsule shell. Matrix capsules are used in particular if a controlled, in particular retarded and/or continuous release of the encapsulated active ingredient is to take place over a defined period, it being possible to establish different release rates and/or different release profiles through appropriate choice of the matrix materials and the capsule sizes, whereas core/shell encapsulations permit in particular short-term releases or else releases over a very long period.

In particular, the inside capsules of the dosage forms according to the invention have a capsule shell or capsule matrix which completely encloses the active ingredient.

In this connection, the capsule shell or capsule matrix material of the inside capsules can in principle be identical or different with regard to the capsule shell material of the outside capsule. Preferably, the capsule shell or capsule matrix material of the inside capsules is designed different with regard to the capsule shell material of the outside capsule.

In particular, the capsule shell or capsule matrix material of the inside capsules can comprise at least one pharmacologically acceptable polymer or consist thereof. According to the invention, synthetic, natural and/or nature-identical polymers and polymerizates are preferably used for this purpose. Particularly preferably, the capsule shell or capsule matrix material of the inside capsules can comprise at least one pharmacologically acceptable polymer or consist thereof, which is selected from the group of (A) polysaccharides, in particular alginates, cellulose and cellulose derivatives, chitosans, starch and starch derivatives, agar agar, carrageenans, pectins, galactomannans, guarans, dextrans, xanthans, glucans and gum Arabic; (B) proteins, in particular gelatin and caseinates; (C) polylactides, in particular homo- and copolymers of lactic acid; (D) amino resins; (B) poly(meth)acrylates; (F) polyureas; (G) polyelectrolytes or polyelectrolyte complexes; (H) waxes; (I) paraffins; (J) colloids and hydrocolloids, in particular based on polysaccharide and/or protein; and mixtures and combinations thereof, particularly preferably from the group of (A) polysaccharides, in particular alginates, cellulose and cellulose derivatives, chitosans, starch and starch derivatives, agar agar, carrageenans, pectins, galactomannans, guarans, dextrans, xanthans, glucans and gum Arabic; (B) proteins, in particular gelatin and caseinates; (C) polylactides, in particular homo- and copolymers of lactic acid; and mixtures and combinations thereof.

In general, the inside capsules of the dosage form according to the invention can be produced by means of dropletization processes, interfacial polycondensation processes, interfacial polyaddition processes, solvent evaporation processes, spray-drying processes or phase separation processes, preferably by means of dropletization processes. According to the invention, preference is given to inside capsules which are obtainable by means of dropletization processes; these inside capsules produce the best results within the context of the present invention. As regards the production process of the capsules, detailed explanations are given below.

In general, the diameter, in particular the average diameter, of the inside capsules is designed to be smaller than the diameter, in particular the average diameter, of the outside capsule at least by one order of magnitude.

In general, in this connection, the ratio of the diameter, in particular of the average diameter, of the inside capsules to the diameter, in particular to the average diameter, of the outside capsule can be at least 1:2, in particular at least 1:5, preferably at least 1:10, particularly preferably at least 1:15, very particularly preferably at least 1:20.

Usually, the ratio of the diameter, in particular of the average diameter, of the inside capsules to the diameter, in particular to the average diameter, of the outside capsule can be up to 1:100 000, in particular up to 1:50 000, preferably up to 1:15 000, particularly preferably up to 1:10 000, very particularly preferably up to 1:5000.

In general, the ratio of the diameter, in particular of the average diameter, of the inside capsules to the diameter, in particular to the average diameter, of the outside capsule can vary in the range from 1:2 to 1:100 000, in particular in the range from 1:5 to 1:50 000, preferably in the range from 1:10 to 1:15 000, particularly preferably in the range from 1:15 to 1:10 000, very particularly preferably in the range from 1:20 to 1:5000.

The diameter, in particular the average diameter, of the inside capsules can vary within wide ranges. Usually, the diameter, in particular the average diameter, of the inside capsules can vary in the range from 0.1 μm to 10 mm, in particular in the range from 1 μm to 8 mm, preferably in the range from 5 μm to 7 mm, particularly preferably in the range from 10 μm to 5 mm, very particularly preferably in the range from 25 μm to 4 mm, even more preferably in the range from 50 μm to 3 mm.

In this connection, the inside capsules can preferably have a monomodal graining and/or size distribution.

The diameter, in particular the average diameter, of the outside capsule can also vary within wide ranges. Usually, the diameter, in particular the average diameter, of the outside capsule can vary in the range from 50 μm to 100 mm, in particular in the range from 100 μm to 75 mm, preferably in the range from 250 μm to 60 mm, particularly preferably in the range from 500 μm to 50 mm, very particularly preferably in the range from 750 μm to 30 mm, even more preferably in the range from 1000 μm to 25 mm.

As explained at the start, the capsules-in-capsule system according to the invention comprises a plurality of inside microcapsules located in an outside capsule. In particular, the outside capsule can comprise at least two inside capsules, in particular at least five inside capsules, preferably at least ten inside capsules, particularly preferably at least 15 inside capsules, very particularly preferably at least 20 inside capsules. Usually, the outside capsule can comprise up to 10 000 inside capsules, in particular up to 5000 inside capsules, preferably up to 1000 inside capsules, particularly preferably up to 500 inside capsules, very particularly preferably up to 100 inside capsules.

As regards the active ingredient 1,8-cineole, the dosage form according to the invention or the inside capsules can thus comprise 1,8-cineole as the sole active ingredient or else—according to an alternative, but less preferred embodiment—can comprise the active ingredient 1,8-cineole at least with at least one further active ingredient, preferably selected from terpenes. According to the invention, it is preferred if the dosage form according to the invention and/or the inside capsules comprise 1,8-cineole as the sole active ingredient.

According to one specific embodiment, the inside capsules comprise 1,8-cineole together with at least one physiologically acceptable carrier (excipient) which is miscible with 1,8-cineole and/or soluble therein, and in particular is liquid at 20° C. and atmospheric pressure. The carrier or excipient is itself not pharmacologically active, but forms a preferably homogeneous mixture or solution with the active ingredient 1,8-cineole. In this way, within the scope of the present invention, a microencapsulation of the active ingredient 1,8-cineole is possible, which would otherwise not be possible with customary microencapsulation methods.

According to one embodiment preferred according to the invention, the carrier or excipient can be selected from the group of fatty oils, preferably triglycerides, particularly preferably medium chain triglycerides (MCT), very particularly preferably triglycerides with C₆-C₁₂-fatty acid radicals.

According to the invention, it is preferred if the carrier or excipient is used in a 1,8-cineole/carrier quantitative ratio in the range from 1000:1 to 1:1000, in particular 100:1 to 1:100, preferably 50:1 to 1:50, particularly preferably 10:1 to 1:10, very particularly preferably 5:1 to 1:2, even more preferably 3:1 to 1:1; in this way, a particularly good microencapsulation of the active ingredient 1,8-cineole is possible.

The term oils as used according to the invention for the above carriers or excipients is a collective name for liquids which cannot be mixed with water. The term fatty oils as used in this connection according to the invention refers specifically to fats, i.e. mixtures of fatty acid triglycerides, which are liquid at room temperature (in particular 20° C.) and atmospheric pressure; in particular, this term refers to esters of the trivalent alcohol glycerol (propane-1,2,3-triol) with three, mostly different, predominantly even-numbered and unbranched aliphatic monocarboxylic acids, the so-called fatty acids. Compounds of this type are also called triglycerides (according to IUPAC recommendation: Triacylglycerins). Triglycerides, synonymously also referred to as glycerol triesters, are thus triple esters of the trivalent alcohol glycerol with three acid molecules, the prefix “tri” referring to three acyl acid radicals esterified with glycerol.

Specifically medium chain triglycerides, as are preferably used according to the invention as carrier or excipient for the active ingredient 1,8-cineole, are in particular semi-synthetic neutral glycerol esters of saturated, generally unbranched monocarboxylic acids of medium chain length (i.e. C₆-C₁₂). In particular, the term medium chain triglycerides refers to mixtures of triglycerides of saturated fatty acids, primarily caprylic acid (octanoic acid) and capric acid (decanoic acid). Medium chain triglycerides can generally be produced from oil which is extracted from the solid and dried part of the endosperm of Cocos nucifera L. and/or from the dried endosperm of Elaeis guineenses Jacq. For further details relating to the term medium chain triglycerides, reference can be made for example to the monograph Ph. Eur. 6^{th} edition, basic work 2008, pages 4224 to 4226, and to the Zeitschrift für Ernährungswis-senschaft, volume 13, book 1/2, 1973, pages 6 ff., D. Sailer et al. “Mitte/kettige Triglyceride—Klinische Physiologie and Anwendung”) [Medium chain triglycerides—clinical physiology and use]).

The active ingredient content (i.e. the 1,8-cineole content) of the dosage form according to the invention can equally vary within wide limits: usually, the dosage form according to the invention comprises the active ingredient 1,8-cineole in absolute amounts of from 10 to 1000 mg, in particular 25 to 750 mg, preferably 50 to 500 mg, particularly preferably 75 to 300 mg. In general, the dosage form according to the invention comprises the active ingredient 1,8-cineole in relative amounts of from 0.01 to 99% by weight, in particular 0.1 to 95% by weight, preferably 1 to 90% by weight, particularly preferably 5 to 80% by weight.

Within the context of the present invention, the dosage form according to the invention is generally present in a gastric juice-resistant, small intestine-soluble form, i.e. the dosage form according to the invention is usually designed to be resistant to gastric juice but soluble in the small intestine. This means that the active ingredient 1,8-cineole is released, in the case of systemic application, only at the actual site of absorption, i.e. the small intestine. For this purpose, the outside capsule and/or the inside capsules can be present in a gastric juice-resistant, small intestine-soluble form, preferably be provided with a gastric juice-resistant, small intestine-soluble coating or shell. Such gastric juice-resistant, but small intestine-soluble coating or shell materials are known per se to the person skilled in the art: for this purpose, for example (meth)acrylic acid polymers, in particular methacrylic, acid/ethyl acrylate copolymers (e.g. Eudragit®), can be used; for further details relating to methacrylic acid/ethyl acrylate copolymers used for this purpose, reference can be made for example to the monograph Ph. Eur., 6^{th} edition, basic work 2008, pages 3215 to 3217. Alternatively, according to one embodiment preferred according to the invention, a mixture of oleic acid, stearic acid and ethyl cellulose can also be used for this purpose as gastric juice-resistant, small intestine-soluble coating or covering.

Further advantageous properties, aspects and features of the present invention arise from the following description of one embodiment shown in the FIGURES. The FIGURE shows:
a diagrammatic cross section through a cineole-containing dosage form according to the invention as per one embodiment of the present invention.

The single FIGURE shows a cineole-containing dosage form 1 according to the invention, intended for peroral application and designed in the form of a capsule, in accordance with one embodiment of the present invention. As the FIGURE representation reveals, the dosage form 1 is designed as a capsules-in-capsule system, where the capsules-in-capsule system has an outer capsule (outside capsule) 2 with an outer capsule shell and a plurality of inner capsules (inside capsules) 3 located in the outer capsule, where the inner capsules 3 are completely enclosed by the capsule shell of the outer capsule 2 and the inner capsules 3 are designed as microcapsules which each contain the active ingredient 1,8-cineole.

For further details relating to the embodiment shown in the FIGURE representation, reference may be made, for the purposes of avoiding unnecessary repetition, to the above statements, which apply accordingly with regard to the FIGURE presentation.

The present invention further provides—according to a second aspect of the present invention—a process for producing a cineole-containing dosage form according to the present invention, which has been described above, where, according to the process of the invention, firstly microcapsules which each contain the active ingredient 1,8-cineole are produced and then in each case a plurality of the microcapsules produced beforehand is completely enclosed by an outer capsule shell and combined and/or integrated to give a capsules-in-capsule system according to the invention with the microcapsules as inner capsules (inside capsules).

As described above, the microcapsules and/or inside capsules can be produced by means of dropletization processes, interfacial polycondensation processes, interfacial polyaddition processes, solvent evaporation processes, spray-drying processes or phase separation processes. In a manner preferred according to the invention, a dropletization process is used for producing the microcapsules and/or inside capsules. The aforementioned processes are per se known in principle to the person skilled in the art, but have hitherto been used neither for the active ingredient 1,8-cineole nor in connection with the overall process according to the invention in combination with the other process steps.

Hitherto, in the prior art, it has not been possible to produce a 1,8-cineole-containing capsules-in-capsule system with 1,8-cineole-containing microcapsules as inner capsules (inside capsules) since 1,8-cineole cannot be processed directly homogeneously to give corresponding microcapsules, especially on account of its relatively high melting point of +1.5° C.

However, the applicant has now found, in a completely surprising and unexpected way, that the production of 1,8-cineole-containing microcapsules, in particular as inner capsules (inside capsules) of a capsules-in-capsule system is possible if the production of the microcapsules is carried out in the presence of a carrier or excipient as described above for the 1,8-cineole, i.e. the microencapsulation of a mixture or of a solution of 1,8-cineole on the one hand and a carrier or excipient of the aforementioned type on the other hand is carried out.

Within the context of the present invention, the microcapsules or inner capsules (inside capsules) containing 1,8-cineole are preferably produced here by means of a dropletization process, where a microcapsule-forming starting material (e.g. alginates) is dropletized in the presence of 1,8-cineole and optionally at least one physiologically acceptable carrier (excipient) which is miscible with 1,8-cineole and/or soluble therein and is liquid at 20° C. and atmospheric pressure, in particular as described above, and solidified to give microcapsules containing 1,8-cineole. For further details in this regard in relation to the carrier or excipient, reference may be made to the statements above in order to avoid unnecessary repetitions.

In the course of the production process according to the invention, in the process step of microencapsulation of the active ingredient 1,8-cineole, the procedure can in particular be as follows:

The active ingredient 1,8-cineole is brought into contact with the carrier or excipient described previously and mixed intimately and/or homogeneously. The resulting mixture or solution of 1,8-cineole and carrier or excipient is then subsequently subjected to microencapsulation.

For this purpose, the liquid to be microencapsulated consisting of 1,8-cineole/carrier or excipient is supplied together with the capsule-forming material (e.g. alginates) under pressure to a suitable nozzle head and dropletized by means of this nozzle head to give microbeads, which can take place for example by means of suitable oscillation, as a consequence of which the jet of liquid leaving the nozzle head is cut and divided into individual segments or small beads, which subsequently, in particular as a consequence of the surface tension, adopt a spherical configuration and are then solidified in a suitable medium (e.g. a suitable ionic solution).

A microencapsulation process which can be used for these purposes is described for example in FR 2 645 439 A1 or in EP 0 391 803 B1, which belongs to the same patent family, and the disclosure of which in each case is hereby incorporated by reference: according to this process, alginate-based microcapsules are provided by a dropletization process, which in the case of the present invention can be used specifically on the microencapsulation of the 1,8-cineole/carrier mixture described above. In this connection, the alginate droplets laden with 1,8-cineole active ingredient are solidified in a suitable ionic liquid (e.g. calcium chloride solution).

According to the invention, of equal suitability for producing the microcapsules or inside capsules is the dropletization process described in WO 93/02785 A1 and DE 41 25 133 A1, the disclosure of which in each case is hereby incorporated by reference. According to this process, alginate-based microcapsules are produced where, for this purpose, alginate-based alginate beads containing the already described 1,8-cineole/carrier mixture are produced from drops of alginate solution dispensed from a nozzle by solidification of the drops by adding this dropwise to an ionic solution and optional subsequent washing of alginate beads removed from the ionic solution, where the alginate solution can be dropletized by vibrational stimulation and the drops are essentially freely mobile until the desired solidification in the ionic solution.

The disclosure of all of the aforementioned documents mentioned in connection with the microencapsulation is hereby incorporated by reference, meaning that reference can be made to the aforementioned documents as regards further details relating to the production of the microcapsules to be produced in the course of the process according to the invention.

The microencapsulation process is thus carried out in a manner known per se to the person skilled in the art, but in particular with the proviso that an above-described solution or mixture of 1,8-cineole and special excipients or carriers are used for the encapsulation.

The process steps of producing the 1,8-cineole-containing active ingredient microcapsules are then followed by the application of the outer shell or outer layer in each case onto and/or around a plurality of the microcapsules produced in this way. This is a process step known per se to the person skilled in the art, meaning that further details in this regard are superfluous.

With regard to further details relating to the process according to the invention, in order to avoid unnecessary repetition, reference can be made to the above statements relating to the dosage form according to the invention, which apply accordingly with regard to the process according to the invention.

The present invention further provides—according to a third aspect of the present invention—the use of a cineole-containing dosage form as described above in accordance with the present invention in human medicine and in veterinary medicine.

Thus, the cineole-containing dosage form according to the invention can be used for example for the prophylactic and/or therapeutic treatment of inflammatory, infection-exacerbated or allergic diseases of the human or animal body.

Equally, for example, the cineole-containing dosage form according to the invention can be used for the prophylactic and/or therapeutic treatment of autoimmune diseases of the human or animal body.

Furthermore, the cineole-containing dosage form according to the invention can be used for the prophylactic and/or therapeutic treatment of colds and flu infections and diseases and infections associated therewith, in particular infections of the upper and lower respiratory tracts, such as in particular rhinitis, sinusitis and bronchopulmonary diseases.

Furthermore, the cineole-containing dosage form according to the invention can be used for the prophylactic and/or therapeutic treatment of respiratory tract diseases, in particular of bronchopulmonary diseases, in particular bronchitis, bronchial asthma and chronic obstructive pulmonary diseases (COPD). In this connection, a comedication with other therapeutics or medicaments, in particular corticosteroids, is also possible.

Equally, the cineole-containing dosage form according to the invention can also be used for the prophylactic and/or therapeutic treatment of inflammatory diseases of the bile ducts, in particular cholecystitis and cholangitis.

Furthermore, the cineole-containing dosage form according to the present invention can also be used for the prophylactic and/or therapeutic treatment of inflammatory diseases of the lower urinary tract, in particular glomerulonephritis, pyelonephritis, cystitis and uritritis.

The cineole-containing dosage form according to the invention can equally be used for the prophylactic and/or therapeutic treatment of inflammatory diseases of the intestine, in particular Crohn's disease and colitis ulcerosa.

Equally, the dosage form according to the invention can also be used for the prophylactic and/or therapeutic treatment of inflammatory or allergic skin diseases, in particular eczemas and dermatitis.

Furthermore, the dosage form according to the invention can also be used for the prophylactic and/or therapeutic treatment of rheumatoid diseases (i.e. rheumatic disease or rheumatism, and also all diseases of the rheumatic category).

Finally, the dosage form according to the invention can also be used for the prophylactic and/or therapeutic treatment of systemically corticosteroid-dependent diseases (i.e. all diseases which have to be treated with systemically administered corticosteroids). This can be explained by the corticosteroid-like pharmacological effect profile of 1,8-cineole.

For all of the uses above, a comedication with at least one further therapeutic agent and/or drug is possible.

Within the context of the use according to the invention, the cineole-containing dosage form, according to the present invention is usually administered in amounts such that the 1,8-cineole is administered in daily doses of from 50 to 3000 mg/diem, in particular 100 to 2000 mg/diem, preferably 200 to 1000 mg/diem, and/or that the 1,8-cineole is provided for administration in a daily dose of from 50 to 3000 mg/diem, in particular 100 to 2000 mg/diem, preferably 200 to 1000 mg/diem.

For further details in this respect relating to the use according to the invention, in order to avoid unnecessary repetition, reference may be made to the above statements relating to the dosage form according to the invention and to the production process according to the invention, which apply accordingly with regard to the use according to the invention.

Finally, the present invention provides—according to a fourth aspect of the present invention—a drug or medicinal product which comprises the cineole-containing dosage form described above in accordance with the present invention.

Within the context of the drug or medicinal product according to the invention, the drug or medicinal product is provided in particular for an administration of 1,8-cineole in a daily dose of from 50 to 3000 mg/diem, in particular 100 to 2000 mg/diem, preferably 200 to 1000 mg/diem.

For further details in this regard relating to the drug or medicinal product according to the invention, in order to avoid unnecessary repetition, reference may be made to the above statements relating to the dosage form according to the invention, the production process according to the invention and the use according to the invention, which apply accordingly as regards the drug or medicinal product according to the invention.

Further embodiments, modifications and variations, and also advantages of the present invention are directly evident and realizable for the person skilled in the art upon reading the description, without, in so doing, departing from the essence of the present invention.

The present invention is illustrated by reference to the following working examples, although these in no way limit the present invention.

### Working Examples

### Production of a Dosage Form According to the Invention

A dosage form according to the invention based on a capsules-in-capsule system is produced as follows:

Firstly, a homogeneous mixture based on 1,8-cineole on the one hand and medium chain triglycerides on the other hand in a weight ratio of about 2:1 is prepared.

On the basis of this 1,8-cineole/triglyceride mixture, alginate-based matrix microcapsules which contain the active ingredient 1,8-cineole are subsequently produced in accordance with the process and apparatus as per FR 2 645 436 A1 by means of the dropletization process described therein.

For this purpose, an aqueous sodium alginate solution which comprises the above-described 1,8-cineole/triglyceride mixture is produced optionally together with customary additives and subsequently this aqueous sodium alginate solution containing 1,8-cineole is dropletized in a manner known per se into a cross-linking solution containing calcium chloride, which moreover can contain customary additives (e.g. surfactants etc.).

Matrix microcapsules containing 1,8-cineole as active ingredient and with alginate as matrix material result. The microcapsules obtained in this way are subsequently purified and optionally isolated.

Alternatively to the procedure described above, it is also possible, in accordance with FR 2 645 439 A1, to firstly produce pure alginate-based matrix microcapsules (i.e. matrix microcapsules without the active ingredient 1,8-cineole), which are subsequently introduced into a solution based on the above-described 1,8-cineole/triglyceride mixture and laden herewith in this way.

The micromatrix capsules laden with the active ingredient 1,8-cineole obtained in this way are then further processed in a manner known per se to give a capsules-in-capsule system according to the invention by surrounding in each case a plurality of the above-described microcapsules with an outside shell.

The outside shell is finally provided with a gastric juice-resistant, but small intestine-soluble coating. For this purpose, for example (meth)acrylic acid polymers, in particular methacrylic acid/ethyl acrylate copolymers (e.g. Eudragit®), can be used. Alternatively to this, a mixture of oleic acid, stearic acid and ethyl cellulose, however, can also be used for this purpose.

### Clinical Application Observations and Stability Studies

The above-described dosage form containing 1,8-cineole as active ingredient according to the present invention based on a capsules-in-capsule system is used in the course of the treatment of bronchial asthma in comedication to corticosteroids.

The comparison used is standard commercial 1,8-cineole-containing capsules which in each case comprise 200 mg of 1,8-cineole per capsule, where the standard commercial capsules have a liquid 1,8-cineole-containing core and an outer, gastric juice-resistant, but small intestine-soluble shell layer.

In the course of the clinical application observations, in each case 15 subjects are treated with standard commercial 1,8-cineole-containing capsules with a triple dose of 200 mg/diem on the one hand and with the dosage form according to the invention with a single dose of 500 mg/diem on the other hand. After treatment for just four days with 1,8-cineole, the lung function (increase in FeV₁, decrease in airway resistance, Raw) is improved in the same way, and in all cases the daily corticosteroid requirement can be reduced by ca. 50% to 60% as a result of an increasing stabilization of the bronchial asthma. Within the context of the dosage form according to the invention, the administration only once daily with an effective dose reduced by 100 mg/diem brings about the same effect as the triple dose of in each case 200 mg/diem with standard commercial capsules which contain the active ingredient not with microencapsulation.

The long-term treatment with 1,8-cineole is tolerated significantly better in the case of the capsules-in-capsule system according to the invention; in particular, side effects, such as gastrointestinal troubles, in particular gastric heaviness, gastric overacidification and reflux, are significantly reduced here.

In the case of the dosage form according to the invention, the storage stability under standardized storage conditions is also improved by more than 70% compared with standard commercial 1,8-cineole-containing capsules and thus optimized.

In the course of the production process according to the invention, moreover, the adjustment of the amount of active ingredient per capsule compared with the production of conventional 1,8-cineole-containing capsules is significantly improved and consequently optimized.

As a result, the dosage form according to the invention based on the 1,8-cineole-containing capsules-in-capsule system leads to an improvement and optimization manufacturability and also the application properties of 1,8-cineole-containing capsule systems.

### Further Clinical Application Observations

The above-described dosage form containing 1,8-cineole as active ingredient according to the present invention based on a capsules-in-capsule system is used in the course of treating Crohn's disease and colitis ulcerosa in comedication to corticosteroids.

The comparison used is in turn standard commercial 1,8-cineole-containing capsules which in each case comprise 200 mg of 1,8-cineole per capsule, where the standard commercial capsules have a liquid core comprising 1,8-cineole and an outer, gastric juice-resistant, but small intestine-soluble shell layer.

In the course of the clinical application observations, in each case 12 subjects are treated with standard commercial 1,8-cineole-containing capsules with a triple dose of 200 mg/diem on the one hand and with the dosage form according to the invention with a single dose of 500 mg/diem on the other hand.

On account of the inflammatory component, in the course of the clinical application it is possible to also treat Crohn's disease and colitis ulcerosa analogously to the treatment of bronchial asthma using the same treatment regime.

Here too, the same positive clinical effect can be brought about with the dosage form according to the invention with the lower dose, which is expressed by the fact that the stool frequency is reduced by almost half. Likewise, here too, the dose of the systemic corticosteroids can be reduced by ca. 60% to 70%. In the context of the dosage form according to the invention, the only once daily administration with an effective dose reduced by 100 mg/diem brings about the same effect as the triple administration of in each case 200 mg/diem with standard commercial capsules which contain the active ingredient not with microencapsulation.

Comparable results are also obtained during the treatment of systemically corticosteroid-dependent rheumatic or rheumatoid diseases: here too, the dose of the systemic corticosteroids be reduced by ca. 50% to 70%, where also in this connection, in the context of the dosage form according to the invention, the only once daily administration with an effective dose reduced by 100 mg/diem brings about the same effect as the triple administration of in each case 200 mg/diem with standard commercial capsules which contain the active ingredient not with microencapsulation.

1-15. (canceled)

16. A cineole-containing dosage form for peroral application in the form of a capsule, where the dosage form is designed as a capsules-in-capsule system,
where the capsules-in-capsule system has an outer capsule (outside capsule) having an outer capsule shell and a plurality of inner capsules (inside capsules) located in the outer capsule, where the inner capsules are completely enclosed by the capsule shell of the outer capsule and the inner capsules are designed as microcapsules which each contain the active ingredient 1,8-cineole and
where the inner capsules comprise 1,8-cineole together with at least one physiologically acceptable carrier which is miscible with 1,8-cineole or soluble therein, and is liquid at 20° C. and atmospheric pressure, where the carrier is selected from the group of triglycerides.

17. The dosage form as claimed in claim 16, wherein the inside capsules are present in the form of matrix capsules or in the form of core/shell capsules.

18. The dosage form as claimed in claim 16, wherein the capsule shell material or the capsule matrix material of the inside capsules comprises at least one pharmacologically acceptable polymer or consists thereof, which polymer is selected from synthetic, natural or nature-identical polymers and polymerizates from the group consisting of (A) polysaccharides, alginates, cellulose and cellulose derivatives, chitosans, starch and starch derivatives, agar agar, carrageenans, pectins, galactomannans, guarans, dextrans, xanthans, glucans and gum Arabic; (B) proteins, gelatin and caseinates; (C) polylactides, homo- and copolymers of lactic acid; (D) amino resins; (E) poly(meth)acrylates; (F) polyureas; (G) polyelectrolytes or polyelectrolyte complexes; (H) waxes; (I) paraffins; (J) colloids and hydrocolloids, colloids and hydrocolloids based on polysaccharide or protein; and mixtures and combinations thereof, particularly preferably from the group of (A) polysaccharides, in particular alginates, cellulose and cellulose derivatives, chitosans, starch and starch derivatives, agar agar, carrageenans, pectins, galactomannans, guarans, dextrans, xanthans, glucans and gum Arabic; (B) proteins, in particular gelatin and caseinates; (C) polylactides, in particular homo- and copolymers of lactic acid; and mixtures and combinations thereof.

19. The dosage form as claimed in claim 16, wherein the inside capsules are obtainable by means of dropletization processes, interfacial polycondensation processes, interfacial polyaddition processes, solvent evaporation processes, spray-drying processes or phase separation processes.

20. The dosage form as claimed in claim 16, wherein the ratio of the diameter of the inside capsules to the diameter of the outside capsule is at least 1:5 and up to 1:100,000.

21. The dosage form as claimed in claim 16, wherein the dosage form comprises 1,8-cineole as the sole active ingredient.

22. The dosage form as claimed in claim 16, wherein the dosage form comprises 1,8-cineole at least with at least one further active ingredient selected from terpenes.

23. The dosage form as claimed in claim 16, wherein the carrier is selected from the group of medium chain triglycerides (MCT) and triglycerides with C₆-C₁₂-fatty acid radicals.

24. The dosage form as claimed in claim 16, wherein the carrier is used in a 1,8-cineole/carrier quantitative ratio in the range from 100:1 to 1:100.

25. The dosage form as claimed in claim 16, wherein the dosage form comprises the active ingredient 1,8-cineole in absolute amounts of from 10 to 1,000 mg

26. The dosage form as claimed in claim 16, wherein the dosage form comprises the active ingredient 1,8-cineole in relative amounts of from 0.01 to 99% by weight.

27. The dosage form as claimed in claim 16, wherein the dosage form is designed to be resistant to gastric juice, but soluble in the small intestine.

28. The dosage form as claimed in claim 16, wherein at least on of the outside capsule and the inside capsules is provided with a gastric juice-resistant, small intestine-soluble coating or shell.

29. A process for producing a cineole-containing dosage form as claimed in claim 16,
where firstly microcapsules which each contain the active ingredient 1,8-cineole together with at least one physiologically acceptable carrier that is miscible with 1,8-cineole or soluble therein, and is liquid at 20° C. and atmospheric pressure are produced, where the carrier is selected from the group of triglycerides, and
where a plurality of the microcapsules produced in this way is completely enclosed by an outer capsule shell and is combined or integrated to give a capsules-in-capsule system with the microcapsules as inner capsules.

30. The process as claimed in claim 29, wherein the production of the microcapsules is carried out by means of dropletization processes, interfacial polycondensation processes, interfacial polyaddition processes, solvent evaporation processes, spray-drying processes or phase separation processes.

31. The process as claimed in claim 29, wherein the microcapsules are produced by means of a dropletization process, where a microcapsule-forming starting material is dropletized in the presence of 1,8-cineole and the carrier (excipient) and solidified to give microcapsules containing 1,8-cineole.

32. A method for the prophylactic or therapeutic treatment of a human or animal body suffering from inflammatory, infection-exacerbated or allergic diseases, the method comprising administering the dosage form as claimed in claim 16 in an efficient amount.

33. The method as claimed in claim 32, wherein the disease to be treated is selected from the group consisting of autoimmune diseases; colds and flu infections and diseases and infections associated therewith, infections of the upper and lower respiratory tracts, rhinitis, sinusitis; respiratory tract diseases, bronchopulmonary diseases, bronchitis, bronchial asthma and chronic obstructive pulmonary diseases (COPD); inflammatory diseases of the bile ducts, cholecystisis and cholangitis; inflammatory diseases of the lower urinary tract, glomerulonephritis, pyelonephritis, cystitis and uritritis; inflammatory diseases of the intestine, Crohn's disease and colitis ulcerosa; inflammatory or allergic skin diseases, eczemas and dermatitis; rheumatoid diseases, rheumatic diseases, rheumatism and diseases of the rheumatic category; systemically corticosteroid-dependent diseases and diseases which are treated with systemically administered corticosteroids.

34. The method as claimed in claim 32, wherein the 1,8-cineole is administered in daily doses of from 50 to 3,000 mg/diem.

35. A drug or medicinal product, comprising the cineole-containing dosage form as claimed in claim 16.

36. The drug or medicinal product as claimed in claim 35, wherein the drug or medicinal product is provided for an administration of 1,8-cineole in a daily dose of from 50 to 3,000 mg/diem.
